(19) United States
(12) Patent Application Publication
(10) Pub. No.: US 2021/0263029 A1
(43) Pub. Date: Aug. 26, 2021

(54) METHOD FOR FULL-RANGE DETECTION OF C-REACTIVE PROTEIN AND CORRESPONDING KIT

(71) Applicants: XIAMEN INNODX BIOTECH CO. LTD, Xiamen, Fujian (CN); XIAMEN UNIVERSITY, Xiamen, Fujian (CN)

(72) Inventors: Zimin Chen, Xiamen (CN); Junhui Xiong, Xiamen (CN); Weiling Xu, Xiamen (CN); Zuxing Weng, Xiamen (CN); Long Wang, Xiamen (CN); Xiaohong Zheng, Xiamen (CN); Xudong Sun, Xiamen (CN); Shengxiang Ge, Xiamen (CN)

(21) Appl. No.: 17/254,227

(22) PCT Filed: May 29, 2019
(86) PCT No.: PCT/CN2019/089015

§ 371 Date: Dec. 18, 2020

(30) Foreign Application Priority Data
Jun. 20, 2018 (CN) 201810634120.5

(51) Int. Cl. G01N 33/543; G01N 33/68; G01N 33/577; G01N 33/535; G01N 33/533; G01N 21/76

(52) U.S. Cl. CPC G01N 33/54393; G01N 33/68; G01N 33/54326; G01N 2333/4737; G01N 33/535; G01N 33/533; G01N 21/76; G01N 33/577

(57) ABSTRACT

The invention provides a kit for full-range detection of C-reactive protein based on chemiluminescence immunoassay and a method for full-range detection of C-reactive protein. The kit comprises an R1 reagent, an M reagent, an R2 reagent, a pre-excitation solution and an excitation solution. The R1 reagent, that is a sample treatment solution, is a 0.5M citric acid solution (pH 3.0-3.5, which is adjusted by disodium hydrogen phosphate dodecahydrate). The present invention also provides a kit for full-range detection of C-reactive protein, which comprises a flat-bottomed plate-type chemiluminescence plate coated with a first antibody, a sample treatment solution, a second antibody labeled with horseradish peroxidase (HRP) or alkaline phosphatase (AP), a color developing solution. The invention also provides a method for full-range detection of C-reactive protein using the kit. Both the first antibody and the second antibody are monoclonal antibodies that can specifically react with C-reactive protein.

### TECHNICAL FIELD

The invention belongs to the technical field of chemiluminescence immunoassays, and specifically relates to a method for full-range detection of C-reactive protein and a corresponding kit.

### BACKGROUND

C-Reactive protein (CRP) is an acute phase reaction protein discovered by Tillet and Francis in 1930 that can react with Streptococcus pneumoniae C polysaccharide in the presence of Ca²⁺ to form a complex; serum CRP is synthesized by hepatocytes under the stimulation of IL-6, IL-2 and TNF, and inflammatory local macrophages are also produced in small amounts. CRP has a molecular weight of about 115KD and consists of five identical unglycosylated polypeptide subunits, each subunit contains 204 amino acids, these subunits are connected by non-covalent bonds to form a cyclic pentamer, and with an interchain disulfide bond, this pentameric protein has remarkable heat resistance and protein degradation resistance.

CRP is widely distributed in the body. In addition to blood, it can be detected in pleural fluid, ascites, pericardial fluid, and joint fluid.

CRP is an important acute reaction protein. It starts to increase at 6-8 h after the occurrence of bacterial infection and reaches a peak at 24-48 h. After the infection is eliminated, its content drops sharply and returns to normal within a week.

The clinical application of CRP is mainly used as a first-choice indicator to identify bacterial or viral infections, as well as to monitor disease changes and postoperative infections, to dynamically observe the efficacy of antibiotics, to guide and monitor treatments and the like. CRP is also related to cardiovascular disease, coronary heart disease, and acute coronary syndrome, in which the level of CRP in patients is often significantly elevated, and the degree of the elevated level is significantly correlated to the degree of coronary artery obstruction, the occurrence and prognosis of the end-event of coronary heart disease, and congestive heart failure. In addition, CRP is also an independent predictor of atrial fibrillation, and there is a certain correlation between serum CRP concentration and hypertension. The systolic and diastolic blood pressure levels of hypertensive patients increase with the increase of serum CRP concentration.

At present, CRP detection method on the market mainly include hypersensitive CRP (hsCRP) detection, conventional CRP detection, and full-range CRP detection. Hypersensitive CRP detection is mainly used to diagnose and predict the occurrence and development of cardiovascular events, while conventional CRP detection is mainly used for bacterial infection, various inflammatory processes, tissue necrosis and tissue damage (such as post-operative damage), as well as screening, monitoring, disease evaluation and efficacy judgment during recovery period. Early conventional CRP detection methods are mainly based on immuno-scattering turbidity or immuno-transmitting turbidity methods, with a detection capacity of more than 5 mg/L, but they are difficult to predict the risk of cardiovascular disease due to the lack of high sensitivity. Subsequent research and development provide an immune enhancement turbidimetric method, which analysis sensitivity is greatly improved, the lower limit of detection can reach 0.02 mg/L. This hypersensitive CRP detection for low concentrations is called hypersensitive CRP detection. With the continuous innovation and improvement of technology, some detection methods can cover the detection linearity of hypersensitivity and full-range CRP at one time, such as chemiluminescence detection methods and immunofluorescence detection methods. The line width of detection can reach 0.02-100 mg/L.

At present, the full-range CRP detection methods usually use the addition of competing free antibodies. For example, US Patent Application Publication No. US 2014/0017712 A1 mentions the use of adding a free monoclonal antibody or an antibody that can compete with coating or labeling. However, such a method increases difficulty in operations such as reagent stability. Chinese Patent Publication No. CN105988003A discloses a method in which the purpose of full-range detection is achieved by using alkali neutralization after acid destruction, but it is still not stable and convenient. Therefore, there still exists a need in the art to improve the method of the full-range CRP detection to realize a full-range CRP detection method in a stable and convenient detection mode.

### Contents of the Invention

The purpose of the present invention is to overcome the defects of the prior art and provide a stable and convenient method for full-range detection of C-reactive protein as well as a corresponding kit.

The technical solution of the present invention is as follows:

In one aspect, the present invention provides a kit for full-range detection of C-reactive protein, which comprises:

an M reagent, comprising 0.5˜1 mg/mL magnetic particles coated with a first antibody, 0.04˜0.06% (w/v) surfactant (the surfactant is optionally Tween-20), and 8˜12% (w/v) sucrose, its solvent is a phosphate buffer with pH=7.0˜8.0; wherein the coating amount of the first antibody is 520 μg/mg magnetic particles;

an R1 reagent, that is a sample treatment solution, which is a citric acid solution with a concentration of 0.1˜1M, pH=3.0˜4.0;

an R2 reagent, comprising acridinium ester coated with a secondary antibody, 0.5-1% casein and 0.5-1% bovine serum albumin, its solvent is phosphate buffer with pH=7.0-8.0, wherein the coating amount of the secondary antibody is 0.3-0.9 μg/μg acridinium ester;

a pre-excitation solution and an excitation solution;

wherein the first antibody and the second antibody are both monoclonal antibodies that can specifically react with C-reactive protein, and the first antibody and the second antibody are directed to different epitopes.

In another aspect, the present invention provides a kit for full-range detection of C-reactive protein, which comprises:

a flat-bottomed plate-type chemiluminescence plate coated with a first antibody, which comprises a plate-type luminescence plate (optionally, 96-well, 384-well or other plate-type luminescence plate), wherein the coating amount of the first antibody is 100˜500 ng/well (optionally 500 ng/well), the coating buffer is a phosphate buffer with pH=7.0˜8.0, the blocking solution is 50 mM phosphate buffer with pH of 7.2-7.4 comprising 5-8% (w/v) blocking serum or blocking protein (the blocking serum is optionally calf serum) and 0.02% (w/v) sodium azide;

a sample treatment solution, which is a citric acid solution with a concentration of 0.1˜1M, pH=3˜4;

a labeling enzyme solution, comprising a secondary antibody labeled with horseradish peroxidase or alkaline phosphatase, and having a labeling amount that 1 mg/mL of the secondary antibody is labeled with horseradish peroxidase or alkaline phosphatase in the same proportion;

a color developing solution: when the labeling enzyme is horseradish peroxidase, the color developing solution comprises a color developing solution A and a color developing solution B, and the color developing solution A is hydrogen peroxide (optionally, the formula of the color developing solution A: 13.6 g of sodium acetate, 1.6 g of citric acid, 0.3 ml of 30% hydrogen peroxide, formulated with distilled water to 500 ml), the color developing solution B is o-phenylenediamine (optionally, the formula of the color developing solution B: 0.2 g of disodium ethylenediaminetetraacetate, 0.95 g of citric acid, 50 ml of glycerol, 9.15 g of tetramethylbenzidine, formulated with distilled water to 500 ml); when the labeling enzyme is alkaline phosphatase, the color developing solution is a commercially available reagent;

wherein the first antibody and the second antibody are both monoclonal antibodies that can specifically react with C-reactive protein, and the first antibody and the second antibody are directed to different epitopes.

In some embodiments, the pH of the citric acid solution is adjusted by disodium hydrogen phosphate dodecahydrate; preferably, the pH of the citric acid solution is 3.0-3.5; and more preferably, the pH of the citric acid solution is 3.2, 3.3, 3.4 or 3.5.

In other embodiments, the concentration of the citric acid is 0.5 mol/L.

In some embodiments, the pre-excitation solution is 1% (w/v) hydrogen peroxide solution, the excitation solution is 1 mol/L sodium hydroxide solution, the first antibody is 10C11, and the second antibody is 14D9-2.

In still other embodiments, the method for preparing the M reagent comprises: the first antibody and the magnetic particles are mixed in 2-morpholineethanesulfonic acid buffer with pH=5.0˜6.0, coated at 25-37° C. for 1-3 h, added with 0.1%-0.5% (w/v) bovine serum albumin phosphate buffer with pH=8.0˜9.0 to perform termination for 1˜3 h, the coated magnetic particles are separated and dispersed in a phosphate buffer with pH=7.0˜8.0, then added with 0.04˜0.06% (w/v) surfactant (the surfactant is optionally Tween-20; in one embodiment, the surfactant is 0.05% (w/v) Tween-20) and 8˜12% (w/v) sucrose (optionally, 10% (w/v) sucrose) to obtain the M reagent;

the method for preparing the R2 reagent comprises: the second antibody and acridinium ester are mixed in a phosphate buffer with pH=8.0˜9.0, coated at 25-37° C. for 1˜3 h, and then added with a Tris buffer comprising 0.1%-0.5% (w/v) bovine serum albumin and having pH=8.0˜9.0 to perform termination for 1˜3 h so as to obtain a stock solution, and the stock solution is diluted with a phosphate buffer having pH=7.0˜8.0 to 1:100˜500 to obtain the R2 reagent.

In other embodiments, the method for preparing the luminescent plate coating source comprises: the coated first antibody is diluted with a phosphate buffer having pH=7.0-8.0 as coating buffer to 100-500 ng/well (optionally, 500 ng/well), added to the luminescent plate, 100 μL per well, incubated at 37° C. for 2 h or 4° C. overnight, the coating buffer is poured out, 200 μL of the blocking solution comprising 5-8% (w/v) calf serum and 0.02% (w/v) sodium azide is used for incubation at 37° C. for 2 h, the liquid in the wells is poured out, the plate is dried and sealed under vacuum with aluminum film, and stored in a dry place at 4° C.;

the method for preparing the labeling enzyme solution comprises: the second antibody and horseradish peroxidase or alkaline phosphatase in ratio of 1:1 are mixed and labeled and dialyzed in a carbonate buffer with pH=9.6, and the dialysis buffer is replaced every 4 hours and replaced for three times, the enzyme-labeled secondary antibody is collected to be a stock solution, and then the stock solution is diluted with a commercially available enzyme diluent to 1:500 to obtain the labeling enzyme solution.

In yet another aspect, the present invention provides a method for full-range detection of C-reactive protein, which is performed using the kit of the present invention, and which comprises:

(1) 20 μL of a sample is taken and added to 100 μL of the R1 reagent to treat the sample;

(2) 50 μL of the M reagent is then added and incubated together for 15 min;

(3) after step (2), washing is performed with a phosphate buffer comprising 0.05˜0.08% (w/v) Tween-20, then 50 μL of the R2 reagent is added and incubated for 10 minutes;

(4) after step (3), washing is performed with a phosphate buffer comprising 0.05˜0.08% (w/v) Tween-20, and 100 μL of the pre-excitation solution is added to perform pre-excitation;

(5) the pre-excitation solution is removed, 100 μL of the excitation solution is then added to perform excitation and detection.

In another aspect, the present invention provides a use of a citric acid solution as a sample treatment solution in manufacture of a kit for full-range detection of C-reactive protein.

In some embodiments, the citric acid solution is a citric acid solution with a concentration of 0.1˜1M, pH=3˜4; preferably, the pH of the citric acid solution is adjusted by disodium hydrogen phosphate dodecahydrate, more preferably, the pH of the citric acid solution is 3.0-3.5, more preferably, the pH of the citric acid solution is 3.2, 3.3, 3.4 or 3.5.

In one aspect, the present invention provides a kit for full-range detection of C-reactive protein (direct chemiluminescence, that is, magnetic particle-chemiluminescence method), which comprises the following components:

an M reagent, comprising 0.5˜1 mg/mL magnetic particles coated with a first antibody, 0.04˜0.06% (w/v) surfactant (the surfactant is optionally Tween-20), and 8˜12% (w/v) sucrose, its solvent is a phosphate buffer with pH=7.0˜8.0; wherein the coating amount of the first antibody is 5˜20 μg/mg magnetic particles;

an R1 reagent, that is a sample treatment solution, which is a citric acid solution with a concentration of 0.1˜1M, pH=3.0˜4.0;

an R2 reagent, comprising acridinium ester coated with a secondary antibody, 0.5-1% casein and 0.5-1% bovine serum albumin, its solvent is phosphate buffer with pH=7.0-8.0, wherein the coating amount of the secondary antibody is 0.3-0.9 μg/μg acridinium ester;

a pre-excitation solution and an excitation solution;

wherein the first antibody and the second antibody are both monoclonal antibodies that can specifically react with C-reactive protein, and the first antibody and the second antibody are directed to different epitopes.

The luminescence mechanism of acridine compounds is: in an alkaline hydrogen peroxide solution, the molecule of acridine compound is attacked by hydrogen peroxide ions to form an unstable peroxy compound, which decomposes into CO₂ and electronically excited N-methyl-acridone, when it returns to its ground state, it emits a photon with a maximum emission wavelength of 430 nm. Surfactants such as Triton X-100, Tween-20, CTAC (hexadecyltrimethylammonium chloride, a cationic surfactant) can enhance luminescence.

In a preferred embodiment of the present invention, the R1 reagent is 0.5M citric acid solution, pH=3˜3.5.

Further preferably, the pH of the R1 reagent is adjusted by disodium hydrogen phosphate dodecahydrate.

In one embodiment, the pH of the citric acid solution is adjusted by disodium hydrogen phosphate dodecahydrate. Preferably, the pH of the citric acid solution is 3.0-3.5, and more preferably, the pH of the citric acid solution is 3.2, 3.3, 3.4 or 3.5.

In yet another embodiment, the concentration of the citric acid is 0.5 mol/L.

Further preferably, the M reagent contains 0.05% Tween-20 and 10% sucrose.

Further preferably, the method for preparing the M reagent comprises: the first antibody and the magnetic particles are mixed in 2-morpholineethanesulfonic acid buffer with pH=5.0˜6.0, coated at 25-37° C. for 1-3 h, added with 0.1%-0.5% (w/v) bovine serum albumin phosphate buffer with pH=8.0˜9.0 to terminate the coating for 1˜3 h, the coated magnetic particles are separated and dispersed in a phosphate buffer with pH=7.0˜8.0, then added with Tween-20 and sucrose to obtain the M reagent.

Further preferably, the method for preparing the R2 reagent comprises: the second antibody and acridinium ester are mixed in a phosphate buffer with pH=8.0˜9.0, coated at 25-37° C. for 1˜3 h, and then added with a Tris buffer comprising 0.1%-0.5% (w/v) bovine serum albumin and having pH=8.0˜9.0 to terminate the coating for 1˜3 h so as to obtain a stock solution, and the stock solution is diluted with a phosphate buffer having pH=7.0˜8.0 to 1:100˜500 to obtain the R2 reagent.

Further preferably, the pre-excitation solution is a 1% (w/v) hydrogen peroxide solution.

Furthermore, the excitation solution is 1 mol/L sodium hydroxide solution.

In another aspect, the present invention provides a kit for full-range detection of C-reactive protein (enzymatic chemiluminescence, namely horseradish peroxidase or alkaline phosphatase plate-type chemiluminescence), which comprises the following components:

a flat-bottomed chemiluminescence plate coated with a first antibody, which comprises a plate-type luminescence plate (optionally, 96-well, 384-well or other plate-type luminescence plate), wherein the coating amount of the first antibody is 100˜500 ng/well (optionally 500 ng/well), the coating buffer is a phosphate buffer with pH=7.0˜8.0, the blocking solution is 50 mM phosphate buffer with pH of 7.2-7.4 comprising 5-8% (w/v) blocking serum or blocking protein (the blocking serum is optionally calf serum) and 0.02% (w/v) sodium azide;

a sample treatment solution, which is a citric acid solution with a concentration of 0.1˜1M, pH=3˜4;

a labeling enzyme solution, comprising a secondary antibody labeled with horseradish peroxidase or alkaline phosphatase, and having a labeling amount that 1 mg/mL of the secondary antibody is labeled with horseradish peroxidase or alkaline phosphatase in the same proportion;

a color developing solution: when the labeling enzyme is horseradish peroxidase, the color developing solution comprises a color developing solution A and a color developing solution B, and the color developing solution A is hydrogen peroxide (13.6 g of sodium acetate, 1.6 g of citric acid, 0.3 ml of 30% hydrogen peroxide, formulated with distilled water to 500 ml), the color developing solution B is o-phenylenediamine (the formula of the color developing solution B: 0.2 g of disodium ethylenediaminetetraacetate, 0.95 g of citric acid, 50 ml of glycerol, 9.15 g of tetramethylbenzidine, formulated with distilled water to 500 ml); when the labeling enzyme is alkaline phosphatase, the color developing solution is a commercially available reagent (Art. No.: 180309-01, purchased from: Xiamen Boson Biotechnology Co., Ltd.).

Detection: an automatic chemiluminescence analyzer (purchased from: Yantai Addcare Biotechnology Co., Ltd.) is used for reading the luminescence values.

The above-mentioned first antibody and second antibody are both monoclonal antibodies that can specifically react with C-reactive protein.

In a preferred embodiment of the present invention, the sample treatment solution is a 0.5 M citric acid solution with a pH of 3 to 3.5.

Further preferably, the pH of the sample treatment solution is adjusted by disodium hydrogen phosphate dodecahydrate.

In one embodiment, the pH of the citric acid solution is adjusted by disodium hydrogen phosphate dodecahydrate. Preferably, the pH of the citric acid solution is 3.0-3.5, and more preferably, the pH of the citric acid solution is 3.2, 3.3, 3.4 or 3.5.

In yet another embodiment, the concentration of the citric acid is 0.5 mol/L.

Further preferably, the flat-bottomed chemiluminescent plate coated with the first antibody comprises 5-8% calf serum and 0.02% sodium azide.

Further preferably, the method for preparing the flat-bottomed chemiluminescent plate coated with the first antibody comprises: the coated first antibody is diluted with a phosphate buffer having pH=7.0-8.0 as coating buffer to 5 μg/mL, i.e., 500 ng/well, added to the luminescent plate, 100 μL per well, incubated at 37° C. for 2 h or 4° C. overnight, the coating buffer is poured out, 200 μL of the blocking solution (5-8% (w/v) calf serum and 0.02% (w/v) sodium azide) is used for incubation at 37° C. for 2 h, the liquid in the wells is poured out, the plate is dried and sealed under vacuum with aluminum film, and stored in a dry place at 4° C.

Further preferably, the method for preparing the labeling enzyme solution comprises: the second antibody and horseradish peroxidase or alkaline phosphatase in ratio of 1:1 are mixed and labeled and dialyzed in a carbonate buffer with pH=9.6, and the dialysis buffer is replaced every 4 hours, for three times, the enzyme-labeled secondary antibody is collected to be the stock solution, and then the stock solution is diluted with a commercially available enzyme diluent to 1:500 to obtain the labeling enzyme solution.

Further preferably, when the labeling enzyme is horseradish peroxidase, the color developing solution A is hydrogen peroxide, and the color developing solution B is o-phenylenediamine; when the labeling enzyme is alkaline phosphatase, the color developing solution is a commercially available reagent.

Furthermore, the content is directly determined by an automatic chemiluminescence analyzer.

The beneficial effect of the present invention is that the detection range of the kit of the present invention can be 0.02 mg/L to 100 mg/L after a sample treatment solution (citric acid solution with a concentration of 0.1˜1M, pH=3˜4) is added in one step during the reaction process of the kit of the present invention, so that the kit can meet the requirements of full-range detection of C-reactive protein.

### BRIEF DESCRIPTION OF THE DRAWINGS

FIG. 1 shows a paired dose-response curve, which is a curve of a calibrator for pairing detection. The left image represents the paired dose-response curve for 10C11-7D9, and the right image represents the paired dose-response curve for 10C11-14D9-2.

FIG. 2 shows a correlation analysis of pairing detection results, which evaluates the correlation between samples and background values. The left image represents the correlation analysis of pairing detection results for 10C11-7D9, and the right image represents the correlation analysis of pairing detection results for 10C11-14D9-2.

### DETAILED EMBODIMENTS OF THE INVENTION

The technical solution of the present invention is further illustrated and described below through specific embodiments.

The reagents were of analytical grade, and unless otherwise specified, they were purchased from Xiamen Xilong Chemical Co., Ltd.

In one embodiment, the kit for full-range detection of C-reactive protein (direct chemiluminescence, that is, magnetic particle chemiluminescence method) of the present invention comprises the following components:

an M reagent, comprising 0.8 mg/mL magnetic particles coated with a first antibody, 0.05% Tween-20 and 10% sucrose, and its solvent was a phosphate buffer with pH=7.5, wherein the coating amount of the first antibody was 12 μg/μg magnetic particles, the magnetic particles were purchased from Thermo Fisher Scientific and were nano-scale superparamagnetic particles with Fe₃O₄ core. The method for preparing the M reagent comprised: the first antibody and magnetic particles were mixed in 2-morpholineethanesulfonic acid buffer with pH=5.5, coated at 32° C. for 1˜3 h, and added with a phosphate buffer solution comprising 0.3% bovine serum albumin and having pH=8.5 to terminate the coating for 2 h, the coated magnetic particles were separated and dispersed in a phosphate buffer solution with pH=7.5, and then added with Tween-20 and sucrose to obtain the M reagent;

an R1 reagent, a citric acid solution with a concentration of 0.5M, pH=3.2, adjusted with disodium hydrogen phosphate dodecahydrate;

an R2 reagent, comprising acridinium ester coated with the secondary antibody, 0.8% casein and 0.8% bovine serum albumin, its solvent was a phosphate buffer with pH=7.5, and the coating amount of the secondary antibody was 12 μg/μg acridinium ester. The method for preparing the R2 reagent comprised: the second antibody and acridinium ester were mixed in a phosphate buffer with pH=8.5, coated at 32° C. for 1˜3 h, added with a Tris buffer solution comprising 0.3% bovine serum albumin and having pH=8.5 to terminate the coating for 2 h so as to obtain a stock solution; the stock solution was diluted with a phosphate buffer having pH=7.5 to 1:300 so as to obtain the R2 reagent;

pre-excitation solution, 1% (w/v) hydrogen peroxide solution;

exciting solution, 1 mol/L sodium hydroxide solution;

the above-mentioned first antibody and second antibody were monoclonal antibodies that could specifically react with C-reactive protein. The first antibody was 10C11 and the second antibody was 14D9-2, both of which were purchased from Xiamen Innovax Biotech CO., Ltd.

The detection method using the above-mentioned kit for full-range detection of C-reactive protein comprised the following steps:

(1) 20 μL of a sample (the sample is a serum or a standard C-reactive protein for preparing antigen standard curve) was taken and added to 100 μL of the R1 reagent to treat the sample;

(2) 50 μL of the M reagent was then added and incubated together for 15 min;

(3) after step (2), washing was performed with a phosphate buffer comprising 0.05% Tween-20, then 50 μL of the R2 reagent was added and incubated for 10 min;

(4) after step (3), washing was performed with a phosphate buffer comprising 0.05˜0.08% (w/v) Tween-20, and 100 μL of the pre-excitation solution is added to perform pre-excitation;

(5) the pre-excitation solution is removed, 100 μL of the excitation solution is added to perform excitation and detection.

In another embodiment, the kit for full-range detection of C-reactive protein (enzymatic chemiluminescence, that is, horseradish peroxidase plate-type chemiluminescence) of the present invention comprised the following components:

a luminescent plate coating source, comprising a 96-well plate luminescent plate, in which the coating amount of the first antibody was 500 ng/well, the coating buffer was a phosphate buffer with pH=7.5, and the blocking solution was 50 mM phosphate buffer with a pH of 7.3 comprising 6% calf serum and 0.02% sodium azide. The method for preparing the luminescent plate coating source comprised: the first antibody was diluted with phosphate buffer having pH=7.5 as coating buffer to 5 μg/mL (i.e., 500 ng/well), added to the luminescent plate, 100 μL per well, incubated at 37° C. for 2 h or at 4° C. overnight, the coating buffer was poured out, 200 μL of the blocking solution comprising 6% calf serum and 0.02% sodium azide was used for incubation at 37° C. for 2 h, the liquid in the wells was poured out, the plate was dried and sealed under vacuum with aluminum film, and stored in a dry place at 4° C.;

a sample treatment solution, which is a citric acid solution with a concentration of 0.5M, pH=3.2, adjusted with disodium hydrogen phosphate dodecahydrate;

a labeling enzyme solution, comprising a secondary antibody labeled with horseradish peroxidase or alkaline phosphatase, and having a labeling amount that 1 mg/mL of the secondary antibody was labeled with horseradish peroxidase and alkaline phosphatase in the same proportion. The method for preparing the labeling enzyme solution comprised: the second antibody and horseradish peroxidase or alkaline phosphatase in ratio of 1:1 were mixed and labeled and dialyzed in a carbonate buffer of pH=9.6, and the dialysis buffer was replaced once every 4 hours, for 3 times, the enzyme-labeled secondary antibody was collected to obtain a stock solution, and then the stock solution was diluted with a commercial enzyme diluent (Cat. No. ED-11, purchased from Beijing Wantai Biopharmaceutical Co., Ltd.) to 1:500 to obtain the labeling enzyme solution;

a color developing solution: when the labeling enzyme was horseradish peroxidase, the color developing solution A was hydrogen peroxide, and the color developing solution B was o-phenylenediamine; when the labeling enzyme was alkaline phosphatase, the color developing solution was a purchased reagent (purchased from: Xiamen Boson Biotechnology Co., Ltd.).

Detection: an automatic chemiluminescence analyzer (purchased from: Yantai Addcare Biotechnology Co., Ltd.) was used for reading the luminescence values.

The above-mentioned first antibody and second antibody were monoclonal antibodies that could specifically react with C-reactive protein. The first antibody was 10C11 and the second antibody was 14D9-2, both of which were purchased from Xiamen Innovax Biotech CO., Ltd.

The detection method using the above-mentioned kit for full-range detection of C-reactive protein comprised the following steps:

(1) 20 μL of a sample was taken and added to 100 μL of the sample treatment solution to treat the sample;

(2) then added to the luminescent plate coating source and incubated together at 37° C. for 40 min;

(3) after step (2), washing was performed for 5 times with a phosphate buffer comprising 0.05% Tween-20, the luminescent plate was turned upside-down till dry, then 100 μL of labeling enzyme solution was added and incubated at 37° C. for 40 min;

(4) after step (3), washing was performed for 5 times with a phosphate buffer comprising 0.05% Tween-20, the luminescent plate was turn upside-down till dry; if the labeling enzyme was horseradish peroxidase, 50 μL of the color developing solution A and 50 μL of the color developing solution B were added, reacted at room temperature for 5 min; if the labeling enzyme was alkaline phosphatase, 100 μL of color developing solution (purchased from: Xiamen Boson Biotechnology Co., Ltd.) was added and reacted at room temperature for 5 min; finally, the automatic chemiluminescence analyzer was used to perform detection and read the luminescence values.

### EXAMPLES

### Example 1

0.1M citric acid and 0.1M glycine of different pH values were selected respectively as treatment solution, and added to the enzyme immunoassay system (the pH range was 2-6) to evaluate the gradiently diluted C-reactive protein antigen. The relative OD values were shown in Tables 1 and 2 below.

TABLE 1
Effects of 0.1M citric acid treatment solutions with different
pH values on the detection of C-reactive protein

| Concentration | | | | | |
|---|---|---|---|---|---|
| (mg/L) | pH = 2 | pH = 3 | pH = 4 | pH = 5 | pH = 6 |
| 100.00 | 0.4090 | 1.4410 | 3.7590 | 0.7350 | 1.0130 |
| 25.00 | 0.5760 | 1.3740 | 3.7620 | 1.0160 | 1.2900 |
| 6.25 | 0.1510 | 0.8930 | 3.7590 | 1.3460 | 1.5250 |
| 1.56 | 0.0430 | 0.3720 | 3.7760 | 2.4110 | 2.8010 |
| 0.39 | 0.0120 | 0.0960 | 3.0050 | 1.5960 | 3.2060 |
| 0.10 | 0.0060 | 0.0240 | 0.9480 | 0.8620 | 2.2130 |
| 0.02 | 0.0100 | 0.0110 | 0.2560 | 0.0640 | 0.2760 |

TABLE 2
Effects of 0.1M glycine treatment solutions with different
pH values on the detection of C-reactive protein

| Concentration | pH = 2 | pH = 3 | pH = 4 | pH = 5 | pH = 6 |
|---|---|---|---|---|---|
| 100 | 3.7910 | 3.7500 | 2.6820 | 3.2820 | 1.7660 |
| 25 | 2.6980 | 3.5760 | 2.3680 | 2.9670 | 2.1690 |
| 6.25 | 0.5270 | 3.0060 | 2.5510 | 3.2550 | 2.3350 |
| 1.56 | 0.1100 | 0.6840 | 2.6910 | 3.3620 | 2.8260 |
| 0.39 | 0.0330 | 0.1780 | 1.1610 | 1.7100 | 1.8980 |
| 0.1 | 0.0190 | 0.0440 | 0.5440 | 0.7650 | 0.6870 |
| 0.02 | 0.0140 | 0.0150 | 0.1090 | 0.1850 | 0.0880 |

Table 1 and Table 2 showed the detection results of the traced antigen of the full-range detection of C-reactive protein in the enzyme immunoassay system when the treatment solution was 0.1M citric acid with different pH values (adjusted to different pH values with disodium hydrogen phosphate dodecahydrate) and the treatment solution was 0.1M glycine with different pH values. The results showed that there was an obvious trend in the detection between pH 3-4, while other pH ranges were not ideal. It could be seen from Tables 1 and 2 that when the range of pH 3-4 was selected as the treatment pH of the treatment solution, the sample detection exhibited a tend from high to low, which was better than other pH ranges. However, the line width of the enzyme immunoassay system was not sufficient for full-range detection, so it was considered that the optimal pH range was 3-4 for chemiluminescence platform exploration, and citric acid was used for subsequent experiments.

### Example 2

Based on Example 1, the improvement and adjustment of citric acid concentration (citric acid concentration of 0.1M, 0.5M, 1M) and pH range (pH3 and pH3.5, pH4) were carried out, the relative linear width of the enzyme immunoassay system was relatively narrow, and the preferred solution was adjusted on chemiluminescence platform. The citric acid solutions with different molar concentrations and pH 3-4 were selected as the treatment solution and added to the chemiluminescence detection system (i.e., magnetic particle chemiluminescence platform) to evaluate the gradiently diluted C-reactive protein antigen. By using citric acid with different pH and different concentration, the gradiently diluted C-reactive protein antigen was treated, and the magnetic particle chemiluminescence platform or the enzymatic horseradish peroxidase chemiluminescence platform was used for detection to obtain the detection results, and the obtained results were made into standard curves. Then, the relative luminescence intensities of the 18 clinical samples collected (from Xiamen Zhongshan Affiliated Hospital and Xijing Hospital) were separately shown in Table 3 and Table 4 below.

TABLE 3
Correlation of the influence of citric acid treatment solution with different pH and different concentration
on the detection of C-reactive protein (magnetic particle chemiluminescence system)

| Citric acid | | | | | | | | | | |
|---|---|---|---|---|---|---|---|---|---|---|
| concentration | | | | | | | | | | |
| (mol/L) | | 0.1 | | | 0.5 | | | 1 | | |
| pH value | | 3 | 3.5 | 4 | 3 | 3.5 | 4 | 3 | 3.5 | 4 |
| Serum serial | Back- | | | | | | | | | |
| number of | ground | | | | | | | | | |
| Zhongshan | value | | | | | | | | | |
| Hospital | (mg/L) | Detection value (mg/L) | | | | | | | | |
| 1257 | 46.70 | 58.17 | 50.68 | 19.67 | 38.70 | 49.77 | 96.64 | 65.72 | 73.63 | 163.59 |
| 1203 | 54.30 | 67.65 | 62.15 | 25.76 | 37.36 | 57.59 | 98.96 | 95.94 | 44.42 | 319.43 |
| 1444 | 170.00 | 106.50 | 120.46 | 37.40 | 87.46 | 137.30 | 189.87 | 273.05 | 209.85 | 79.81 |
| 1315 | 183.00 | 144.27 | 146.56 | 44.94 | 140.15 | 181.43 | 257.77 | 373.53 | 470.72 | 342.03 |
| 1432 | 136.00 | 78.22 | 89.66 | 22.38 | 76.33 | 102.71 | 132.89 | 182.84 | 200.50 | 88.86 |
| 1333 | 110.00 | 99.11 | 100.68 | 45.29 | 62.90 | 109.99 | 186.08 | 165.51 | 65.75 | 186.25 |
| 1478 | 70.30 | 46.69 | 56.99 | 10.28 | 41.24 | 47.21 | 83.52 | 65.98 | 132.74 | 152.31 |
| 1233 | 62.30 | 53.84 | 60.32 | 16.85 | 44.19 | 54.97 | 102.16 | 88.10 | 104.26 | 154.66 |
| 1207 | 84.80 | 97.89 | 75.66 | 17.15 | 79.43 | 69.73 | 113.89 | 107.25 | 120.61 | 91.72 |
| 1210 | 40.10 | 43.45 | 43.21 | 10.23 | 29.06 | 36.09 | 71.60 | 46.09 | 63.54 | 67.85 |
| 1231 | 36.90 | 60.52 | 59.45 | 45.53 | 37.01 | 49.67 | 99.61 | 77.69 | 17.84 | 193.95 |
| 1338 | 33.70 | 39.83 | 39.97 | 6.90 | 30.62 | 35.97 | 69.69 | 47.56 | 5.99 | 124.40 |
| 1223 | 24.20 | 30.60 | 32.36 | 3.34 | 23.25 | 31.46 | 82.90 | 40.27 | 6.61 | 72.51 |
| 1230 | 29.00 | 28.35 | 32.70 | 12.25 | 22.82 | 26.86 | 60.01 | 38.28 | 27.95 | 106.24 |
| 1349 | 16.80 | 19.46 | 24.65 | 8.17 | 15.58 | 19.75 | 56.57 | 29.44 | 12.92 | 88.97 |
| 1317 | 12.10 | 16.12 | 20.92 | 12.24 | 12.03 | 16.52 | 37.35 | 20.29 | 16.66 | 40.57 |
| 1316 | 5.40 | 9.94 | 6.74 | 12.49 | 6.26 | 7.82 | 19.74 | 18.29 | 23.27 | 2.77 |
| 1324 | 9.10 | 10.52 | 11.43 | 17.12 | 8.79 | 7.84 | 24.26 | 13.17 | 19.98 | 39.97 |
| Serum serial | Back- | | | | | | | | | |
| number of | ground | | | | | | | | | |
| Zhongshan | value | | | | | | | | | |
| Hospital | (Log10) | Detection value (Log10) | | | | | | | | |
| 1257 | 1.67 | 1.76 | 1.70 | 1.29 | 1.59 | 1.70 | 1.99 | 1.82 | 1.87 | 2.21 |
| 1203 | 1.73 | 1.83 | 1.79 | 1.41 | 1.57 | 1.76 | 2.00 | 1.98 | 1.65 | 2.50 |
| 1444 | 2.23 | 2.03 | 2.08 | 1.57 | 1.94 | 2.14 | 2.28 | 2.44 | 2.32 | 1.90 |
| 1315 | 2.26 | 2.16 | 2.17 | 1.65 | 2.15 | 2.26 | 2.41 | 2.57 | 2.67 | 2.53 |
| 1432 | 2.13 | 1.89 | 1.95 | 1.35 | 1.88 | 2.01 | 2.12 | 2.26 | 2.30 | 1.95 |
| 1333 | 2.04 | 2.00 | 2.00 | 1.66 | 1.80 | 2.04 | 2.27 | 2.22 | 1.82 | 2.27 |
| 1478 | 1.85 | 1.67 | 1.76 | 1.01 | 1.62 | 1.67 | 1.92 | 1.82 | 2.12 | 2.18 |
| 1233 | 1.79 | 1.73 | 1.78 | 1.23 | 1.65 | 1.74 | 2.01 | 1.94 | 2.02 | 2.19 |
| 1207 | 1.93 | 1.99 | 1.88 | 1.23 | 1.90 | 1.84 | 2.06 | 2.03 | 2.08 | 1.96 |
| 1210 | 1.60 | 1.64 | 1.64 | 1.01 | 1.46 | 1.56 | 1.85 | 1.66 | 1.80 | 1.83 |
| 1231 | 1.57 | 1.78 | 1.77 | 1.66 | 1.57 | 1.70 | 2.00 | 1.89 | 1.25 | 2.29 |
| 1338 | 1.53 | 1.60 | 1.60 | 0.84 | 1.49 | 1.56 | 1.84 | 1.68 | 0.78 | 2.09 |
| 1223 | 1.38 | 1.49 | 1.51 | 0.52 | 1.37 | 1.50 | 1.92 | 1.61 | 0.82 | 1.86 |
| 1230 | 1.46 | 1.45 | 1.51 | 1.09 | 1.36 | 1.43 | 1.78 | 1.58 | 1.45 | 2.03 |
| 1349 | 1.23 | 1.29 | 1.39 | 0.91 | 1.19 | 1.30 | 1.75 | 1.47 | 1.11 | 1.95 |
| 1317 | 1.08 | 1.21 | 1.32 | 1.09 | 1.08 | 1.22 | 1.57 | 1.31 | 1.22 | 1.61 |
| 1316 | 0.73 | 1.00 | 0.83 | 1.10 | 0.80 | 0.89 | 1.30 | 1.26 | 1.37 | 0.44 |
| 1324 | 0.96 | 1.02 | 1.06 | 1.23 | 0.94 | 0.89 | 1.38 | 1.12 | 1.30 | 1.60 |
| r² | | 0.9304 | 0.9586 | 0.2907 | 0.9692 | 0.9615 | 0.9179 | 0.9251 | 0.5942 | 0.4972 |

TABLE 4
Correlation of the influence of citric acid treatment solution with different
pH and different concentration on the detection of C-reactive protein
(enzymatic horseradish peroxidase chemiluminescence system)

| Citric acid | | | | | | | | | | |
|---|---|---|---|---|---|---|---|---|---|---|
| concentration | | | | | | | | | | |
| (mol/L) | | 0.1 | | | 0.5 | | | 1 | | |
| pH value | | 3 | 3.5 | 4 | 3 | 3.5 | 4 | 3 | 3.5 | 4 |
| Serum | | | | | | | | | | |
| serial | Back- | | | | | | | | | |
| number of | ground | | | | | | | | | |
| Zhongshan | value | | | | | | | | | |
| Hospital | (mg/L) | Detection value (mg/L) | | | | | | | | |
| 1242 | 20 | 27.77 | 139.53 | 37.45 | 37.13 | 17.22 | 140.83 | 82.22 | 92.16 | 9.14 |
| 1351 | 5 | 34.19 | 163.47 | 20.06 | 13.03 | 3.15 | 53.52 | 25.86 | 34.85 | 5.10 |
| 1402 | 3.8 | 15.56 | 89.20 | 21.63 | 9.55 | 3.51 | 37.49 | 24.61 | 31.38 | 1.56 |
| 1417 | 25.8 | 59.31 | 289.25 | 30.77 | 54.35 | 15.76 | 221.95 | 73.38 | 110.76 | 7.75 |
| 1408 | 33.2 | 59.31 | 348.65 | 43.69 | 66.34 | 24.84 | 136.92 | 133.23 | 151.15 | 10.03 |
| 1341 | 38.1 | 76.55 | 501.37 | 37.72 | 71.94 | 34.57 | 68.66 | 159.38 | 352.94 | 10.32 |
| 1255 | 45.7 | 64.17 | 234.53 | 42.15 | 83.93 | 39.37 | 137.79 | 195.34 | 231.96 | 11.19 |
| 1247 | 55.6 | 51.16 | 142.44 | 23.93 | 91.89 | 27.36 | 121.47 | 97.02 | 82.81 | 11.30 |
| 1276 | 62.3 | 82.81 | 302.86 | 22.94 | 94.97 | 55.71 | 92.95 | 206.66 | 386.77 | 12.87 |
| 1365 | 65.4 | 64.67 | 406.95 | 34.06 | 113.07 | 47.36 | 129.91 | 303.71 | 240.53 | 12.23 |
| 1404 | 140 | 104.87 | 405.49 | 87.16 | 209.22 | 73.61 | 132.60 | 239.11 | 264.62 | 15.46 |
| 1246 | 13.2 | 27.49 | 125.45 | 27.00 | 19.99 | 5.67 | 125.97 | 54.08 | 60.48 | 9.04 |
| 1239 | 14.6 | 42.23 | 125.20 | 21.44 | 20.84 | 8.52 | 130.21 | 52.57 | 123.92 | 6.15 |
| 1382 | 133 | 85.53 | 592.49 | 70.10 | 155.30 | 90.13 | 100.33 | 424.02 | 506.85 | 19.91 |
| 1393 | 136 | 83.84 | 444.01 | 32.82 | 211.47 | 69.50 | 92.66 | 163.08 | 187.86 | 18.68 |
| 1484 | 70.3 | 59.54 | 970.42 | 102.66 | 105.58 | 43.79 | 85.37 | 421.22 | 383.87 | 21.00 |
| 1277 | 18.4 | 32.57 | 91.97 | 56.87 | 29.44 | 12.78 | 168.98 | 110.16 | 115.56 | 8.94 |
| 1493 | 67.7 | 72.98 | 336.93 | 307.01 | 104.80 | 59.61 | 74.96 | 393.77 | 340.69 | 17.50 |
| Serum | | | | | | | | | | |
| serial | Back- | | | | | | | | | |
| number of | ground | | | | | | | | | |
| Zhongshan | value | | | | | | | | | |
| Hospital | (Log10) | Detection value (Log10) | | | | | | | | |
| 1242 | 1.30 | 1.44 | 2.14 | 1.57 | 1.57 | 1.24 | 2.15 | 1.91 | 1.96 | 0.96 |
| 1351 | 0.70 | 1.53 | 2.21 | 1.30 | 1.11 | 0.50 | 1.73 | 1.41 | 1.54 | 0.71 |
| 1402 | 0.58 | 1.19 | 1.95 | 1.34 | 0.98 | 0.54 | 1.57 | 1.39 | 1.50 | 0.19 |
| 1417 | 1.41 | 1.77 | 2.46 | 1.49 | 1.74 | 1.20 | 2.35 | 1.87 | 2.04 | 0.89 |
| 1408 | 1.52 | 1.77 | 2.54 | 1.64 | 1.82 | 1.40 | 2.14 | 2.12 | 2.18 | 1.00 |
| 1341 | 1.58 | 1.88 | 2.70 | 1.58 | 1.86 | 1.54 | 1.84 | 2.20 | 2.55 | 1.01 |
| 1255 | 1.66 | 1.81 | 2.37 | 1.62 | 1.92 | 1.60 | 2.14 | 2.29 | 2.37 | 1.05 |
| 1247 | 1.75 | 1.71 | 2.15 | 1.38 | 1.96 | 1.44 | 2.08 | 1.99 | 1.92 | 1.05 |
| 1276 | 1.79 | 1.92 | 2.48 | 1.36 | 1.98 | 1.75 | 1.97 | 2.32 | 2.59 | 1.11 |
| 1365 | 1.82 | 1.81 | 2.61 | 1.53 | 2.05 | 1.68 | 2.11 | 2.48 | 2.38 | 1.09 |
| 1404 | 2.15 | 2.02 | 2.61 | 1.94 | 2.32 | 1.87 | 2.12 | 2.38 | 2.42 | 1.19 |
| 1246 | 1.12 | 1.44 | 2.10 | 1.43 | 1.30 | 0.75 | 2.10 | 1.73 | 1.78 | 0.96 |
| 1239 | 1.16 | 1.63 | 2.10 | 1.33 | 1.32 | 0.93 | 2.11 | 1.72 | 2.09 | 0.79 |
| 1382 | 2.12 | 1.93 | 2.77 | 1.85 | 2.19 | 1.95 | 2.00 | 2.63 | 2.70 | 1.30 |
| 1393 | 2.13 | 1.92 | 2.65 | 1.52 | 2.33 | 1.84 | 1.97 | 2.21 | 2.27 | 1.27 |
| 1484 | 1.85 | 1.77 | 2.99 | 2.01 | 2.02 | 1.64 | 1.93 | 2.62 | 2.58 | 1.32 |
| 1277 | 1.26 | 1.51 | 1.96 | 1.75 | 1.47 | 1.11 | 2.23 | 2.04 | 2.06 | 0.95 |
| 1493 | 1.83 | 1.86 | 2.53 | 2.49 | 2.02 | 1.78 | 1.87 | 2.60 | 2.53 | 1.24 |
| r² | | 0.7954 | 0.4898 | 0.2703 | 0.9759 | 0.9495 | 0.1002 | 0.8034 | 0.7264 | 0.8121 |

Tables 3 and 4 showed that when three pH ranges of 3, 3.5 and 4 were fixed and different citric acid concentrations (0.1M, 0.5M and 1M) were used, the luminescent platform was used to detect the traced antigen of the full-range C-reactive protein and evaluate 18 samples. It was found that the citric acid with concentration of 0.5M and pH 3-3.5 showed better results. It could be seen from Table 3 that under the above concentration and pH, the correlation between serums was relative better between pH 3 to 3.5; 0.5M citric acid was preferred, the fluctuation was relatively smaller between pH 3 to 3.5 for 0.5M citric acid, and thus it was considered relatively stable.

### Example 3

0.5M citric acid was selected to further optimize and refine the pH concentration (pH 2.8-4). 0.5M citric acid of different pH was used as the treatment solution to treat the gradiently diluted C-reactive protein antigen, and the magnetic particle chemiluminescence platform or the enzymatic horseradish peroxidase chemiluminescence platform was used for detection to obtain the detection results; and then the results were made into standard curves. The collected 18 clinical samples were then detected, and the detection results were shown in Table 5 and Table 6.

TABLE 5
Correlation of the influence of 0.5M citric acid treatment solutions with different pH
on the detection of C-reactive protein (magnetic particle chemiluminescence system)

| Citric acid | | | | | | | | | |
|---|---|---|---|---|---|---|---|---|---|
| concentration | | | | | | | | | |
| (mol/L) | | 0.5 | | | | | | | |
| pH value | | 2.8 | 3.0 | 3.2 | 3.4 | 3.5 | 3.6 | 3.8 | 4 |
| Serum | | | | | | | | | |
| serial | Back- | | | | | | | | |
| number of | ground | | | | | | | | |
| Zhongshan | value | | | | | | | | |
| Hospital | (mg/L) | Detection value (mg/L) | | | | | | | |
| 1257 | 46.70 | 41.51 | 38.70 | 34.66 | 34.23 | 49.77 | 39.04 | 39.02 | 96.64 |
| 1203 | 54.30 | 52.57 | 37.36 | 42.20 | 37.53 | 57.59 | 51.86 | 47.60 | 98.96 |
| 1444 | 170.00 | 98.97 | 87.46 | 86.84 | 72.81 | 137.30 | 78.51 | 64.71 | 189.87 |
| 1315 | 183.00 | 180.00 | 140.15 | 136.04 | 110.94 | 181.43 | 113.01 | 81.42 | 257.77 |
| 1432 | 136.00 | 75.33 | 76.33 | 72.26 | 66.97 | 102.71 | 74.05 | 58.84 | 132.89 |
| 1333 | 110.00 | 78.86 | 62.90 | 63.58 | 58.17 | 109.99 | 69.18 | 62.38 | 186.08 |
| 1478 | 70.30 | 51.60 | 41.24 | 39.64 | 39.12 | 47.21 | 40.94 | 44.16 | 83.52 |
| 1233 | 62.30 | 44.84 | 44.19 | 41.87 | 43.44 | 54.97 | 45.01 | 39.10 | 102.16 |
| 1207 | 84.80 | 101.81 | 79.43 | 71.45 | 55.36 | 69.73 | 54.71 | 46.83 | 113.89 |
| 1210 | 40.10 | 29.66 | 29.06 | 27.17 | 26.77 | 36.09 | 37.45 | 36.37 | 71.60 |
| 1231 | 36.90 | 45.18 | 37.01 | 38.04 | 33.17 | 49.67 | 39.54 | 42.19 | 99.61 |
| 1338 | 33.70 | 32.44 | 30.62 | 27.45 | 26.63 | 35.97 | 38.25 | 34.35 | 69.69 |
| 1223 | 24.20 | 23.28 | 23.25 | 21.48 | 22.13 | 31.46 | 20.15 | 37.00 | 82.90 |
| 1230 | 29.00 | 25.81 | 22.82 | 20.91 | 26.46 | 26.86 | 29.64 | 34.47 | 60.01 |
| 1349 | 16.80 | 15.64 | 15.58 | 16.75 | 17.43 | 19.75 | 23.79 | 30.96 | 56.57 |
| 1317 | 12.10 | 10.63 | 12.03 | 13.21 | 15.00 | 16.52 | 21.95 | 23.37 | 37.35 |
| 1316 | 5.40 | 5.91 | 6.26 | 7.11 | 8.13 | 7.82 | 11.94 | 13.43 | 19.74 |
| 1324 | 9.10 | 8.11 | 8.79 | 9.96 | 10.39 | 7.84 | 14.53 | 17.05 | 24.26 |
| Serum | | | | | | | | | |
| serial | Back- | | | | | | | | |
| number of | ground | | | | | | | | |
| Zhongshan | value | | | | | | | | |
| Hospital | (Log10) | Detection value (Log10) | | | | | | | |
| 1257 | 1.67 | 1.62 | 1.59 | 1.54 | 1.53 | 1.70 | 1.59 | 1.59 | 1.99 |
| 1203 | 1.73 | 1.72 | 1.57 | 1.63 | 1.57 | 1.76 | 1.71 | 1.68 | 2.00 |
| 1444 | 2.23 | 2.00 | 1.94 | 1.94 | 1.86 | 2.14 | 1.89 | 1.81 | 2.28 |
| 1315 | 2.26 | 2.26 | 2.15 | 2.13 | 2.05 | 2.26 | 2.05 | 1.91 | 2.41 |
| 1432 | 2.13 | 1.88 | 1.88 | 1.86 | 1.83 | 2.01 | 1.87 | 1.77 | 2.12 |
| 1333 | 2.04 | 1.90 | 1.80 | 1.80 | 1.76 | 2.04 | 1.84 | 1.80 | 2.27 |
| 1478 | 1.85 | 1.71 | 1.62 | 1.60 | 1.59 | 1.67 | 1.61 | 1.65 | 1.92 |
| 1233 | 1.79 | 1.65 | 1.65 | 1.62 | 1.64 | 1.74 | 1.65 | 1.59 | 2.01 |
| 1207 | 1.93 | 2.01 | 1.90 | 1.85 | 1.74 | 1.84 | 1.74 | 1.67 | 2.06 |
| 1210 | 1.60 | 1.47 | 1.46 | 1.43 | 1.43 | 1.56 | 1.57 | 1.56 | 1.85 |
| 1231 | 1.57 | 1.65 | 1.57 | 1.58 | 1.52 | 1.70 | 1.60 | 1.63 | 2.00 |
| 1338 | 1.53 | 1.51 | 1.49 | 1.44 | 1.43 | 1.56 | 1.58 | 1.54 | 1.84 |
| 1223 | 1.38 | 1.37 | 1.37 | 1.33 | 1.35 | 1.50 | 1.30 | 1.57 | 1.92 |
| 1230 | 1.46 | 1.41 | 1.36 | 1.32 | 1.42 | 1.43 | 1.47 | 1.54 | 1.78 |
| 1349 | 1.23 | 1.19 | 1.19 | 1.22 | 1.24 | 1.30 | 1.38 | 1.49 | 1.75 |
| 1317 | 1.08 | 1.03 | 1.08 | 1.12 | 1.18 | 1.22 | 1.34 | 1.37 | 1.57 |
| 1316 | 0.73 | 0.77 | 0.80 | 0.85 | 0.91 | 0.89 | 1.08 | 1.13 | 1.30 |
| 1324 | 0.96 | 0.91 | 0.94 | 1.00 | 1.02 | 0.89 | 1.16 | 1.23 | 1.38 |

| | | | | | | | | |
|---|---|---|---|---|---|---|---|---|
| r² | 0.9547 | 0.9692 | 0.9672 | 0.9786 | 0.9615 | 0.9471 | 0.9319 | 0.9179 |

TABLE 6
Correlation of the influence of 0.5M citric acid treatment solution with different pH on the
detection of C-reactive protein (enzymatic horseradish peroxidase chemiluminescence system)

| Citric acid | | | | | | | | | |
|---|---|---|---|---|---|---|---|---|---|
| concentration | | | | | | | | | |
| (mol/L) | | 0.5 | | | | | | | |
| pH value | | 2.8 | 3.0 | 3.2 | 3.4 | 3.5 | 3.6 | 3.8 | 4 |
| Serum | | | | | | | | | |
| serial | Back- | | | | | | | | |
| number of | ground | | | | | | | | |
| Zhongshan | value | | | | | | | | |
| Hospital | (mg/L) | Detection value (mg/L) | | | | | | | |
| 1445 | 170 | 17.65 | 65.14 | 122.17 | 118.01 | 101.83 | 133.13 | 87.17 | 28.26 |
| 1449 | 140 | 18.45 | 58.83 | 120.32 | 138.13 | 139.69 | 137.80 | 113.82 | 22.61 |
| 1283 | 114 | 13.50 | 33.13 | 74.65 | 125.50 | 87.12 | 111.72 | 150.29 | 29.15 |
| 1238 | 114 | 18.20 | 53.46 | 94.57 | 157.07 | 130.74 | 136.98 | 174.20 | 28.40 |
| 1465 | 73.1 | 15.85 | 31.78 | 59.66 | 115.18 | 104.39 | 119.16 | 136.63 | 23.71 |
| 1366 | 65.4 | 8.13 | 23.87 | 45.86 | 83.41 | 54.86 | 58.97 | 86.38 | 22.05 |
| 1303 | 58.8 | 9.23 | 24.63 | 55.07 | 88.79 | 57.88 | 79.32 | 52.87 | 12.73 |
| 1487 | 52.2 | 5.90 | 15.02 | 33.21 | 73.56 | 58.32 | 83.29 | 105.08 | 30.43 |
| 1345 | 42.3 | 3.41 | 10.48 | 18.21 | 33.29 | 38.14 | 37.83 | 65.16 | 28.49 |
| 1454 | 33.2 | 3.67 | 13.92 | 25.27 | 54.27 | 38.88 | 43.03 | 57.80 | 36.47 |
| 1461 | 25.8 | 2.74 | 9.24 | 20.37 | 37.56 | 28.74 | 50.86 | 69.94 | 32.99 |
| 1394 | 18.8 | 3.07 | 11.64 | 20.62 | 42.19 | 26.63 | 33.52 | 82.36 | 33.42 |
| 1470 | 9.2 | 1.70 | 4.39 | 8.60 | 14.50 | 15.27 | 17.11 | 32.14 | 41.04 |
| 1407 | 8.1 | 0.39 | 1.94 | 7.01 | 8.82 | 6.54 | 7.64 | 10.36 | 37.76 |
| 1370 | 5.9 | 0.36 | 1.89 | 4.58 | 11.98 | 6.72 | 6.71 | 6.75 | 27.83 |
| Serum | | | | | | | | | |
| serial | Back- | | | | | | | | |
| number of | ground | | | | | | | | |
| Zhongshan | value | | | | | | | | |
| Hospital | (Log 10) | Detection value (Log10) | | | | | | | |
| 1445 | 2.23 | 1.25 | 1.81 | 2.09 | 2.07 | 2.01 | 2.12 | 1.94 | 1.45 |
| 1449 | 2.15 | 1.27 | 1.77 | 2.08 | 2.14 | 2.15 | 2.14 | 2.06 | 1.35 |
| 1283 | 2.06 | 1.13 | 1.52 | 1.87 | 2.10 | 1.94 | 2.05 | 2.18 | 1.46 |
| 1238 | 2.06 | 1.26 | 1.73 | 1.98 | 2.20 | 2.12 | 2.14 | 2.24 | 1.45 |
| 1465 | 1.86 | 1.20 | 1.50 | 1.78 | 2.06 | 2.02 | 2.08 | 2.14 | 1.37 |
| 1366 | 1.82 | 0.91 | 1.38 | 1.66 | 1.92 | 1.74 | 1.77 | 1.94 | 1.34 |
| 1303 | 1.77 | 0.97 | 1.39 | 1.74 | 1.95 | 1.76 | 1.90 | 1.72 | 1.10 |
| 1487 | 1.72 | 0.77 | 1.18 | 1.52 | 1.87 | 1.77 | 1.92 | 2.02 | 1.48 |
| 1345 | 1.63 | 0.53 | 1.02 | 1.26 | 1.52 | 1.58 | 1.58 | 1.81 | 1.45 |
| 1454 | 1.52 | 0.56 | 1.14 | 1.40 | 1.73 | 1.59 | 1.63 | 1.76 | 1.56 |
| 1461 | 1.41 | 0.44 | 0.97 | 1.31 | 1.57 | 1.46 | 1.71 | 1.84 | 1.52 |
| 1394 | 1.27 | 0.49 | 1.07 | 1.31 | 1.63 | 1.43 | 1.53 | 1.92 | 1.52 |
| 1470 | 0.96 | 0.23 | 0.64 | 0.93 | 1.16 | 1.18 | 1.23 | 1.51 | 1.61 |
| 1407 | 0.91 | -0.41 | 0.29 | 0.85 | 0.95 | 0.82 | 0.88 | 1.02 | 1.58 |
| 1370 | 0.77 | -0.44 | 0.28 | 0.66 | 1.08 | 0.83 | 0.83 | 0.83 | 1.44 |

| | | | | | | | | |
|---|---|---|---|---|---|---|---|---|
| r² | 0.9206 | 0.9468 | 0.9591 | 0.9154 | 0.9401 | 0.9204 | 0.7299 | 0.2197 |

Tables 5 and 6 showed that when the optimal citric acid concentration previously explored was used, the pH concentration was carefully explored, disodium hydrogen phosphate dodecahydrate was selected to adjust different pH values (including pH 2.8 to 4), and the luminescence platform was used to detect the traced antigen of full-range C-reactive protein. It was found that the test samples of pH (3.0-3.5) showed better correlation, in which the magnetic particle chemiluminescence platform was the best at pH 3.4, and showed the best linear detection result.

In the above pH range and in combination with the results in Table 5, the pH was in the range of 3.0 to 3.5, the C-reactive protein single serum had the linear correlation r² of above 0.96, the final preferred condition was 0.5M citric acid with pH (3.4), and the correlation in detection of 18 serum samples was above 0.97. From the results in Table 6, the enzymatic horseradish peroxidase luminescence platform showed the best linear detection results at pH 3.2. The correlation of 15 serum samples was above 0.959.

### Example 4

The optimized detection system was used to detect gradiently diluted C-reactive protein antigen to make a standard curve, then the collected 47 clinical samples were detected, their concentration values were calculated through the standard curve and subjected to the correlation evaluation against the clinical background values, and the results were shown in Table 7 below (magnetic particle chemiluminescence platform):

TABLE 7

| Serum serial | | | | |
|---|---|---|---|---|
| number | Background | | Detection | |
| of Zhongshan | value | | value | |
| Hospital | (mg/L) | Log10 | (mg/L) | Log10 |
| 1 | 10.59 | 1.02 | 10.99 | 1.04 |
| 2 | 16.95 | 1.23 | 14.88 | 1.17 |
| 3 | 107.75 | 2.03 | 112.29 | 2.05 |
| 4 | 23.77 | 1.38 | 16.99 | 1.23 |
| 5 | 11.88 | 1.07 | 12.97 | 1.11 |
| 6 | 37.34 | 1.57 | 27.23 | 1.44 |
| 7 | 14.8 | 1.17 | 15.41 | 1.19 |
| 8 | 65.23 | 1.81 | 71.81 | 1.86 |
| 9 | 64.93 | 1.81 | 49.28 | 1.69 |
| 10 | 135.45 | 2.13 | 225.39 | 2.35 |
| 11 | 38.85 | 1.59 | 25.73 | 1.41 |
| 12 | 81.04 | 1.91 | 102.13 | 2.01 |
| 13 | 37.34 | 1.57 | 21.58 | 1.33 |
| 14 | 15.4 | 1.19 | 11.87 | 1.07 |
| 15 | 10.88 | 1.04 | 12.81 | 1.11 |
| 16 | 15.09 | 1.18 | 16.10 | 1.21 |
| 17 | 6.65 | 0.82 | 6.85 | 0.84 |
| 18 | 9 | 0.95 | 8.20 | 0.91 |
| 19 | 69.29 | 1.84 | 40.24 | 1.60 |
| 20 | 85.55 | 1.93 | 90.29 | 1.96 |
| 21 | 9.42 | 0.97 | 10.08 | 1.00 |
| 22 | 14.11 | 1.15 | 15.54 | 1.19 |
| 23 | 33 | 1.52 | 23.67 | 1.37 |
| 24 | 50.97 | 1.71 | 35.66 | 1.55 |
| 25 | 28.42 | 1.45 | 28.40 | 1.45 |
| Serum serial | | | | |
| number | | | | |
| of Xijing | | | | |
| Hospital | | | | |
| 1 | 1.5 | 0.18 | 2.11 | 0.32 |
| 2 | 1.32 | 0.12 | 2.29 | 0.36 |
| 3 | 4.24 | 0.63 | 5.57 | 0.75 |
| 4 | 0.285 | −0.55 | 0.66 | −0.18 |
| 5 | 0.737 | −0.13 | 1.18 | 0.07 |
| 6 | 5.62 | 0.75 | 6.60 | 0.82 |
| 7 | 3.63 | 0.56 | 4.65 | 0.67 |
| 8 | 1.37 | 0.14 | 2.44 | 0.39 |
| 9 | 1.39 | 0.14 | 2.03 | 0.31 |
| 10 | 37 | 1.57 | 21.02 | 1.32 |
| 11 | 1.4 | 0.15 | 2.49 | 0.40 |
| 12 | 0.159 | −0.80 | 0.34 | −0.46 |
| 13 | 2.87 | 0.46 | 3.94 | 0.60 |
| 14 | 44.3 | 1.65 | 31.67 | 1.50 |
| 15 | 45 | 1.65 | 26.10 | 1.42 |
| 16 | 17.4 | 1.24 | 16.19 | 1.21 |
| 17 | 60.1 | 1.78 | 30.51 | 1.48 |
| 18 | 3.02 | 0.48 | 5.27 | 0.72 |
| 19 | 0.665 | −0.18 | 1.33 | 0.12 |
| 20 | 15.4 | 1.19 | 12.34 | 1.09 |
| 21 | 0.381 | −0.42 | 0.93 | −0.03 |
| 22 | 1.81 | 0.26 | 2.87 | 0.46 |

Through correlation evaluation, the correlation equation of the two was y=0.8151x+0.2179, and the correlation coefficient r²=0.9692, indicating that the two had a good correlation.

The optimized detection system was used to detect gradiently diluted C-reactive protein antigen to make a standard curve, then the collected 73 clinical samples were detected, their concentration values were calculated through the standard curve and subjected to the correlation evaluation against the clinical background values, and the results were shown in Table 8 below (enzymatic horseradish peroxidase chemiluminescence platform):

TABLE 8

| Serum serial | | | | |
|---|---|---|---|---|
| number | Background | | Detection | |
| of Zhongshan | value | | value | |
| Hospital | (mg/L) | Log10 | (mg/L) | Log10 |
| 1 | 20 | 1.30 | 32.42 | 1.51 |
| 2 | 5 | 0.70 | 5.37 | 0.73 |
| 3 | 3.8 | 0.58 | 5.90 | 0.77 |
| 4 | 22.8 | 1.36 | 46.23 | 1.66 |
| 5 | 25.8 | 1.41 | 40.60 | 1.61 |
| 6 | 33.2 | 1.52 | 53.73 | 1.73 |
| 7 | 38.1 | 1.58 | 67.42 | 1.83 |
| 8 | 45.7 | 1.66 | 73.19 | 1.86 |
| 9 | 62.3 | 1.79 | 89.68 | 1.95 |
| 10 | 65.4 | 1.82 | 74.84 | 1.87 |
| 11 | 140 | 2.15 | 155.47 | 2.19 |
| 12 | 110 | 2.04 | 140.33 | 2.15 |
| 13 | 13.2 | 1.12 | 12.42 | 1.09 |
| 14 | 14.6 | 1.16 | 22.56 | 1.35 |
| 15 | 133 | 2.12 | 176.93 | 2.25 |
| 16 | 45 | 1.65 | 93.78 | 1.97 |
| 17 | 65.4 | 1.82 | 111.15 | 2.05 |
| 18 | 70.3 | 1.85 | 92.69 | 1.97 |
| 19 | 18.4 | 1.26 | 27.67 | 1.44 |
| 20 | 67.7 | 1.83 | 82.16 | 1.91 |
| 21 | 170 | 2.23 | 203.55 | 2.31 |
| 22 | 170 | 2.23 | 228.36 | 2.36 |
| 23 | 140 | 2.15 | 216.73 | 2.34 |
| 24 | 182 | 2.26 | 555.17 | 2.74 |
| 25 | 41.7 | 1.62 | 60.82 | 1.78 |
| 26 | 43.1 | 1.63 | 57.47 | 1.76 |
| 27 | 33.2 | 1.52 | 44.20 | 1.65 |
| 28 | 42.3 | 1.63 | 48.68 | 1.69 |
| 29 | 31.3 | 1.50 | 42.16 | 1.62 |
| 30 | 86.1 | 1.94 | 179.86 | 2.25 |
| 31 | 58.8 | 1.77 | 100.40 | 2.00 |
| 32 | 39.6 | 1.60 | 59.01 | 1.77 |
| 33 | 21.8 | 1.34 | 46.29 | 1.67 |
| 34 | 41.7 | 1.62 | 59.99 | 1.78 |
| 35 | 33.3 | 1.52 | 73.34 | 1.87 |
| 36 | 58.6 | 1.77 | 104.54 | 2.02 |
| 37 | 57.8 | 1.76 | 76.28 | 1.88 |
| 38 | 29 | 1.46 | 43.97 | 1.64 |
| 39 | 39.6 | 1.60 | 32.97 | 1.52 |
| 40 | 30.2 | 1.48 | 38.47 | 1.59 |
| Serum serial | | | | |
| number | | | | |
| of Xijing | | | | |
| Hospital | | | | |
| 1 | 58.6 | 1.77 | 110.22 | 2.04 |
| 2 | 26.3 | 1.42 | 28.64 | 1.46 |
| 3 | 24.2 | 1.38 | 22.55 | 1.35 |
| 4 | 5.6 | 0.75 | 7.98 | 0.90 |
| 5 | 7 | 0.85 | 9.95 | 1.00 |
| 6 | 9.1 | 0.96 | 10.88 | 1.04 |
| 7 | 7.8 | 0.89 | 10.32 | 1.01 |
| 8 | 6.9 | 0.84 | 7.85 | 0.89 |
| 9 | 6.5 | 0.81 | 10.58 | 1.02 |
| 10 | 12.1 | 1.08 | 19.77 | 1.30 |
| 11 | 16.8 | 1.23 | 25.96 | 1.41 |
| 12 | 42.3 | 1.63 | 50.42 | 1.70 |
| 13 | 114 | 2.06 | 179.29 | 2.25 |
| 14 | 6.9 | 0.84 | 5.40 | 0.73 |
| 15 | 17.1 | 1.23 | 21.13 | 1.32 |
| 16 | 58.6 | 1.77 | 102.90 | 2.01 |
| 17 | 213 | 2.33 | 418.66 | 2.62 |
| 18 | 20.3 | 1.31 | 35.05 | 1.54 |
| 19 | 170 | 2.23 | 196.84 | 2.29 |
| 20 | 140 | 2.15 | 187.56 | 2.27 |
| 21 | 114 | 2.06 | 152.75 | 2.18 |
| 22 | 114 | 2.06 | 144.88 | 2.16 |
| 23 | 77.9 | 1.89 | 185.85 | 2.27 |
| 24 | 73.1 | 1.86 | 97.58 | 1.99 |
| 25 | 65.4 | 1.82 | 71.60 | 1.85 |
| 26 | 58.8 | 1.77 | 81.52 | 1.91 |
| 27 | 52.2 | 1.72 | 48.59 | 1.69 |
| 28 | 33.2 | 1.52 | 34.49 | 1.54 |
| 29 | 25.8 | 1.41 | 37.18 | 1.57 |
| 30 | 18.8 | 1.27 | 30.58 | 1.49 |
| 31 | 9.2 | 0.96 | 12.20 | 1.09 |
| 32 | 8.1 | 0.91 | 7.77 | 0.89 |
| 33 | 5.9 | 0.77 | 5.73 | 0.76 |

Through correlation evaluation, the correlation equation between the two was y=1.056x+0.0619, and the correlation coefficient r²=0.9506, indicating that the two had a good correlation.

### Example 5

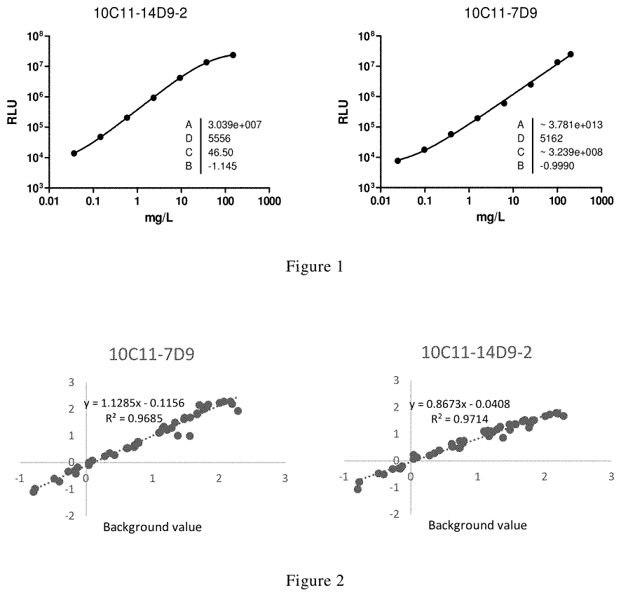

The optimized detection system and the reagents for acid-treatment and alkali-neutralization as mentioned in the patent application with publication number CN105988003A were used to detect the gradiently diluted C-reactive protein antigen so as to make standard curves, and then the collected 48 clinical samples were detected and their concentration values were calculated through the standard curves and subjected to the correlation evaluated against the clinical background values, the performance difference between the two was evaluated, and the results were shown in FIGS. 1 and 2 (magnetic particle chemiluminescence platform).

In comparison of line width of the traced antigen, the two reagents could meet the market demands (0.02-100 mg/L), and the two reagents showed equivalent performance in evaluation of sample correlation.

It could be seen that the detection range of the kit of the present invention could reach 0.02 mg/L to 100 mg/L after the sample treatment solution (citric acid solution with a concentration of 0.1 to 1 M, pH=3 to 4) was added in one step in the reaction process of the kit of the present invention, so that the kit met the requirements of full-range detection of C-reactive protein.

The above are only preferred examples of the present invention, so the scope of implementation of the present invention cannot be limited accordingly. That is, equivalent changes and modifications made according to the scope of the present invention and the contents of the specification should still fall within the scope covered by the present invention.

What is claimed is:

1. A kit for full-range detection of C-reactive protein, which comprises:
an M reagent, comprising 0.5˜1 mg/mL magnetic particles coated with a first antibody, 0.04˜0.06% (w/v) surfactant (the surfactant is optionally Tween-20), and 8˜12% (w/v) sucrose, its solvent is a phosphate buffer with pH=7.0˜8.0; wherein the coating amount of the first antibody is 5˜20 μg/mg magnetic particles;
an R1 reagent, that is a sample treatment solution, which is a citric acid solution with a concentration of 0.1˜1M, pH=3.0˜4.0;
an R2 reagent, comprising acridinium ester coated with a secondary antibody, 0.5-1% casein and 0.5-1% bovine serum albumin, its solvent is a phosphate buffer with pH=7.0-8.0, wherein the coating amount of the secondary antibody is 0.3-0.9 μg/μg acridinium ester;
a pre-excitation solution and an excitation solution;
wherein the first antibody and the second antibody are both monoclonal antibodies that can specifically react with C-reactive protein, and the first antibody and the second antibody are directed to different epitopes.

2. A kit for full-range detection of C-reactive protein, which comprises:
a flat-bottomed plate-type chemiluminescence plate coated with a first antibody, which comprises a plate-type luminescence plate (optionally, 96-well, 384-well or other plate-type luminescence plate), wherein the coating amount of the first antibody is 100˜500 ng/well (optionally 500 ng/well), the coating buffer is a phosphate buffer with pH=7.0˜8.0, the blocking solution is 50 mM phosphate buffer with pH of 7.2-7.4 comprising 5-8% (w/v) blocking serum or blocking protein (the blocking serum is optionally calf serum) and 0.02% (w/v) sodium azide;
a sample treatment solution, which is a citric acid solution with a concentration of 0.1˜1M, pH=3˜4;
a labeling enzyme solution, comprising a secondary antibody labeled with horseradish peroxidase or alkaline phosphatase, and having a labeling amount that 1 mg/mL of the secondary antibody is labeled with horseradish peroxidase or alkaline phosphatase in the same proportion;
a color developing solution: when the labeling enzyme is horseradish peroxidase, the color developing solution comprises a color developing solution A and a color developing solution B, and the color developing solution A is hydrogen peroxide (optionally, the formula of the color developing solution A: 13.6 g of sodium acetate, 1.6 g of citric acid, 0.3 ml of 30% hydrogen peroxide, formulated with distilled water to 500 ml), the color developing solution B is o-phenylenediamine (optionally, the formula of the color developing solution B: 0.2 g of disodium ethylenediaminetetraacetate, 0.95 g of citric acid, 50 ml of glycerol, 9.15 g of tetramethylbenzidine, formulated with distilled water to 500 ml); when the labeling enzyme is alkaline phosphatase, the color developing solution is a commercially available reagent;
wherein the first antibody and the second antibody are both monoclonal antibodies that can specifically react with C-reactive protein, and the first antibody and the second antibody are directed to different epitopes.

3. The kit according to claim 1 or 2, wherein the pH of the citric acid solution is adjusted by disodium hydrogen phosphate dodecahydrate, preferably, the pH of the citric acid solution is 3.0-3.5, more preferably, the pH of the citric acid solution is 3.2, 3.3, 3.4 or 3.5.

4. The kit according to any one of claims 1 to 3, wherein the concentration of the citric acid is 0.5 mol/L.

5. The kit according to any one of claims 1 to 4, wherein the pre-excitation solution is 1% (w/v) hydrogen peroxide solution, and the excitation solution is 1 mol/L sodium hydroxide solution, optionally, the first antibody is 10C11 and the second antibody is 14D9-2.

6. The kit according to any one of claims 1 to 5, wherein:
the method for preparing the M reagent comprises: the first antibody and the magnetic particles are mixed in 2-morpholineethanesulfonic acid buffer with pH=5.0˜6.0, coated at 25-37° C. for 1-3 h, added with 0.1%˜0.5% (w/v) bovine serum albumin phosphate buffer with pH=8.0˜9.0 to perform termination for 1˜3 h, the coated magnetic particles are separated and dispersed in a phosphate buffer with pH=7.0˜8.0, then added with 0.04˜0.06% (w/v) surfactant (the surfactant is optionally Tween-20; in one embodiment, the surfactant is 0.05% (w/v) Tween-20) and 8˜12% (w/v) sucrose (optionally, 10% (w/v) sucrose) to obtain the M reagent;
the method for preparing the R2 reagent comprises: the second antibody and acridinium ester are mixed in a phosphate buffer with pH=8.0˜9.0, coated at 25-37° C. for 1˜3 h, and then added with a Tris buffer comprising 0.1%˜0.5% (w/v) bovine serum albumin and having pH=8.0˜9.0 to perform termination for 1˜3 h so as to obtain a stock solution, and the stock solution is diluted with a phosphate buffer having pH=7.0˜8.0 to 1:100˜500 to obtain the R2 reagent.

7. The kit according to any one of claims 1 to 6, wherein:
the method for preparing the luminescent plate coating source comprises: the coated first antibody is diluted with a phosphate buffer having pH=7.0-8.0 as coating buffer to 100-500 ng/well (optionally, 500 ng/well), added to the luminescent plate, 100 μL per well, incubated at 37° C. for 2 h or 4° C. overnight, the coating buffer is poured out, 200 μL of the blocking solution comprising 5-8% (w/v) calf serum and 0.02% (w/v) sodium azide is used for incubation at 37° C. for 2 h, the liquid in the wells is poured out, the plate is dried and sealed under vacuum with aluminum film, and stored in a dry place at 4° C.;
the method for preparing the labeling enzyme solution comprises: the second antibody and horseradish peroxidase or alkaline phosphatase in ratio of 1:1 are mixed and labeled and dialyzed in a carbonate buffer with pH=9.6, and the dialysis buffer is replaced every 4 hours and replaced for three times, the enzyme-labeled secondary antibody is collected to be a stock solution, and then the stock solution is diluted with a commercially available enzyme diluent to 1:500 to obtain the labeling enzyme solution.

8. A method for full-range detection of C-reactive protein, which is performed by using the kit according to any one of claims 1 to 7, comprising:
(1) 20 μL of a sample is taken and added to 100 μL of the R1 reagent to treat the sample;
(2) 50 μL of the M reagent is then added and incubated together for 15 min;
(3) after step (2), washing is performed with a phosphate buffer comprising 0.05˜0.08% (w/v) Tween-20, then 50 μL of the R2 reagent is added and incubated for 10 minutes;
(4) after step (3), washing is performed with a phosphate buffer comprising 0.05˜0.08% (w/v) Tween-20, and 100 μL of the pre-excitation solution is added to perform pre-excitation;
(5) the pre-excitation solution is removed, 100 μL of the excitation solution is then added to perform excitation and detection.

9. Use of a citric acid solution as a sample treatment solution in manufacture of a kit for full-range detection of C-reactive protein.

10. The use according to claim 9, wherein the citric acid solution is a citric acid solution with a concentration of 0.1˜1M, pH=3˜4; preferably, the pH of the citric acid solution is adjusted by disodium hydrogen phosphate dodecahydrate, more preferably, the pH of the citric acid solution is 3.0-3.5, and more preferably, the pH of the citric acid solution is 3.2, 3.3, 3.4 or 3.5.
